(19) United States
(12) Patent Application Publication
(10) Pub. No.: US 2024/0277962 A1
(43) Pub. Date: Aug. 22, 2024

(54) USER-CONTROLLABLE ENDOTRACHEAL TUBE STYLET

(71) Applicant: THE REGENTS OF THE UNIVERSITY OF CALIFORNIA, Oakland, CA (US)

(72) Inventors: Andrew Vahabzadeh-Hagh, La Jolla, CA (US); David Bracken, San Francisco, CA (US)

(21) Appl. No.: 18/570,479

(22) PCT Filed: Jun. 15, 2022
(86) PCT No.: PCT/US2022/072968

§ 371 Date: Dec. 14, 2023

Related U.S. Application Data
(60) Provisional application No. 63/212,053, filed on Jun. 17, 2021.

(51) Int. Cl. A61M 16/04; A61M 25/01

(52) U.S. Cl. CPC A61M 16/0488; A61M 25/0102

(57) ABSTRACT

Disclosed are articulatable endotracheal tube stylet devices. systems and methods for endotracheal intubation and interrogation. In some aspects. an endotracheal tube stylet apparatus includes a housing: a handle comprising a lever arm that is coupled to the housing: a staple shaft that is coupled to the housing: and an articulation mechanism that extends from the housing through and out of an aperture of the staple shaft and operable to bend upon control by movement of the lever arm of the handle.

### CROSS-REFERENCE TO RELATED APPLICATIONS

This patent document claims priorities and benefits of U.S. Provisional Application No. 63/212,053, titled “USER-CONTROLLABLE ENDOTRACHEAL TUBE STYLET” and filed on Jun. 17, 2021. The entire content of the aforementioned patent application is incorporated by reference as part of the disclosure of this patent document.

### TECHNICAL FIELD

This patent document relates to endotracheal intubation devices and procedures.

### BACKGROUND

The trachea is a cartilaginous tube between the larynx and the bronchi of the lungs that allows air to pass into an out of the lungs during breathing. The larynx closes, serving to protect the trachea and lungs, when fluid and food are ingested. The structure of the trachea includes multiple horseshoe-like rings, which are joined vertically by overlying ligaments, and by the trachealis muscle at their ends posteriorly. The larynx includes the epiglottis, a valve-like structure that further protects the airway during swallowing.

When a patient cannot breathe on their own, intubation is necessary. Endotracheal intubation is the insertion of a breathing tube into the trachea. This may then be connected to a ventilation machine to ventilate a patient that cannot maintain such function on their own. To assist with endotracheal intubation, insertion of a breathing tube, a stylet is most commonly used.

### SUMMARY

Disclosed are articulatable endotracheal tube stylet devices and systems and methods for their use in endotracheal intubation and interrogation. In some aspects, an endotracheal tube stylet apparatus includes a housing; a handle comprising a lever that is coupled to the housing; a shaft that is coupled to the housing; and an articulation mechanism that extends from the housing through and out of an aperture of the shaft and operable to bend upon control by movement of the lever component.

In some aspects, an endotracheal tube stylet apparatus includes a housing; a handle that is coupled to the housing; a shaft that is coupled to the housing; and an articulation mechanism comprising a bendable element and a string, the articulation mechanism extending from the housing through and out of an aperture of the shaft and operable to bend upon control by movement of the handle, wherein the string is coupled to the handle and to a distal end of the bendable element, such that, upon the movement of the handle, the string is pulled to cause a bending of the bendable element along an exposed region of the articulation mechanism outside of the aperture of the shaft.

In some aspects, a method for endotracheal intubation includes providing an endotracheal tube stylet that comprises a housing, a handle that is operably coupled to the housing, a shaft that is coupled to the housing, and an articulation mechanism that is operably coupled to the handle and able to bend in a plurality of bending positions upon movement of the handle; inserting the endotracheal tube stylet into a endotracheal tube; inserting the endotracheal tube that surrounds the endotracheal tube stylet into an airway of a subject; moving the handle of the endotracheal tube stylet to adjust the articulation mechanism to be in a bent position among the plurality of bending positions; and removing the endotracheal tube stylet from the endotracheal tube after the endotracheal tube is inserted in the airway as guided by the endotracheal tube stylet.

The subject matter described in this patent document can be implemented in specific ways that provide one or more of the following features.

### BRIEF DESCRIPTION OF THE DRAWINGS

FIG. 1 shows a diagram of an example embodiment of the endotracheal tube stylet device in accordance with the present technology.

FIGS. 2A and 2B show schematics depicting an example embodiment of an ergonomic handle of the example endotracheal tube stylet device shown in FIG. 1.

FIGS. 3A and 3B show schematics depicting an example embodiment of a housing component of the example endotracheal tube style device shown in FIG. 1.

FIG. 4 shows a diagram of an example embodiment of the endotracheal tube stylet device in accordance of the present technology, featuring another example embodiment of a housing for the device.

FIGS. 5A and 5B show images of an example endotracheal tube stylet shown in FIG. 1 inserted in a standard endotracheal tube and usable for endotracheal intubation.

### DETAILED DESCRIPTION

Endotracheal tubes are used to provide mechanical ventilation for patients undergoing surgical procedures that require general anesthesia and for patients with various forms of respiratory failure. Not considering short term intubation, 1 million U.S. residents require invasive mechanical ventilation per year. Considering outpatient and inpatient surgeries performed daily, this number is far greater (e.g., over 24 million).

During a medical procedure, an endotracheal tube is placed in the airway typically under direct visualization using a rigid laryngoscopy blade to expose the vocal cords and airway. Presently, visualization is difficult to obtain and is often only partial at best. As such, many patients can be difficult to visualize their anatomy using existing endotracheal tube devices, and for some patients with certain limitations such as poor mouth opening and/or neck mobility, this can make airway exposure a lot more difficult. Furthermore, endotracheal tubes are placed in the airway over a rigid stylet that is pre-bent to a configuration favorable for airway access. In many cases, the pre-bent configuration (bend) of the stylet is not appropriate for the patient and either has to be adjusted during the medical procedure, which increases the difficulties for physicians and the risks for patients, or additional medical equipment is required in order to safely and efficiently intubate the patient. For example, insertion and removal of a conventional endotracheal tube in the trachea can result in laryngeal scarring and stenosis after the patient is extubated; and laryngeal scarring can be very difficult to successfully treat. Additionally, other structures may be injured in this process including parts of the face, oral cavity, oropharynx and hypopharynx. Even worse, if the patient is apneic and the airway is unable to be timely accessed, hypoxic injury and even death may result. As such, there is a need to improve the endotracheal tube technology to meet the needs of all users and beneficiaries while improving success on first attempt and minimizing risk.

Here we propose an endotracheal tube stylet device that can articulate (e.g., controllably bend) at the command of user input in real time, e.g., so that the endotracheal tube stylet never needs to be removed and is adjusted in real-time during its use (e.g., in a medical procedure); for example, the endotracheal tube stylet device is adjusted as the airway is approached to achieve perfect access to the airway every time in an atraumatic fashion. Such a device could greatly improve the safety and efficiency of endotracheal intubation.

Disclosed are articulatable endotracheal tube stylet devices, systems and methods for endotracheal intubation and interrogation. In some aspects, an endotracheal tube stylet device is disclosed that comprises a frame; a handle that is moveably coupled to the frame; and an articulation mechanism that is at least partially disposed outside of the frame and operable to be inserted into an endotracheal tube and be used to intubate a patient with user control by bending upon the user moving the handle.

In various implementations of the articulatable endotracheal tube stylet device embodiments described herein, the device allows for real time adjustments to make endotracheal intubation safer, more successful and more efficient. The disclosed endotracheal tube stylet device is configured to be strong enough to generate the forces needed to bend endotracheal tubes of any composition and size with minimal user force. For example, a user can adjust the degree of bend with his/her finger in an easy to use, ergonomic, and intuitive fashion. In some embodiments, for example, the endotracheal tube stylet device can be configured to accommodate endotracheal tube sizes 5.0 to 8.5. In some embodiments, for example, the endotracheal tube stylet device can be used with the full range of pediatric and adult endotracheal tube sizes, regardless of their composition or manufacture specifications. In some embodiments, for example, the endotracheal tube stylet device can be made of recyclable materials and designed for single-use (e.g., disposable) or multi-use applications. In implementations, for example, the endotracheal tube stylet device can be used with any or all traditional intubation devices, e.g., including but not limited to standard laryngoscopes and video laryngoscopes.

In various implementations, for example, the endotracheal tube stylet allows a user (e.g., physician) to adjust the tip of the endotracheal tube to provide anterior flexion to reach and intubate patients more easily. For example, in implementations, the endotracheal tube stylet can be entirely confined within the endotracheal tube and adjust the curvature of the tube itself. This is highly advantageous over conventional endotracheal tube devices especially those that rely on the bougie technique (e.g., the Seldinger technique), where a distal tip is placed deep in the trachea and the endotracheal tube is then advanced over this guide. Moreover, the disclosed endotracheal tube stylet device requires less material in the mouth (e.g., reducing discomfort to patients) and brings the user closer to the airway, and thus is safer and less damaging for adjacent oropharyngeal and laryngeal tissues. The disclosed endotracheal tube stylet device is configured to be simple and easy to use, e.g., with ergonomics very similar to traditional intubation while also being cost-efficient, recyclable, and/or operable for single and/or multi-use.

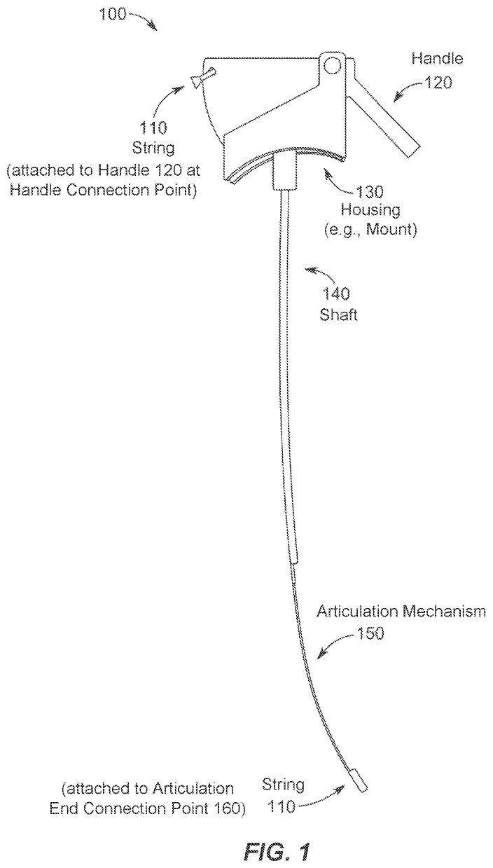

FIG. 1 shows a diagram of an example embodiment of the endotracheal tube stylet device 100 in accordance with the disclosed technology. The device 100 includes a housing 130 that is coupled to a handle 120 and a shaft 140, from which an articulation mechanism 150 extends outward, which is controllably moveable by user-operation of the handle 120.

The articulation mechanism 150 is structured to include an elastically bendable element, which can include a metal spring component (e.g., a spring steel) that is capable of elastically bending or a bendable polymer component. In some embodiments, for example, the elastically bendable metal component includes a metal spring, such as a high carbon steel spring, an alloy steel spring, or another metal material spring. In some embodiments, for example, the elastically bendable polymer component can include acrylic or other bendable polymer. In some embodiments, the elastically bendable element includes a combination of a bendable metal and a bendable polymer. In various embodiments, the elastically bendable element of the articulation mechanism 150 can be configured as a rod, tube, or other elongated component.

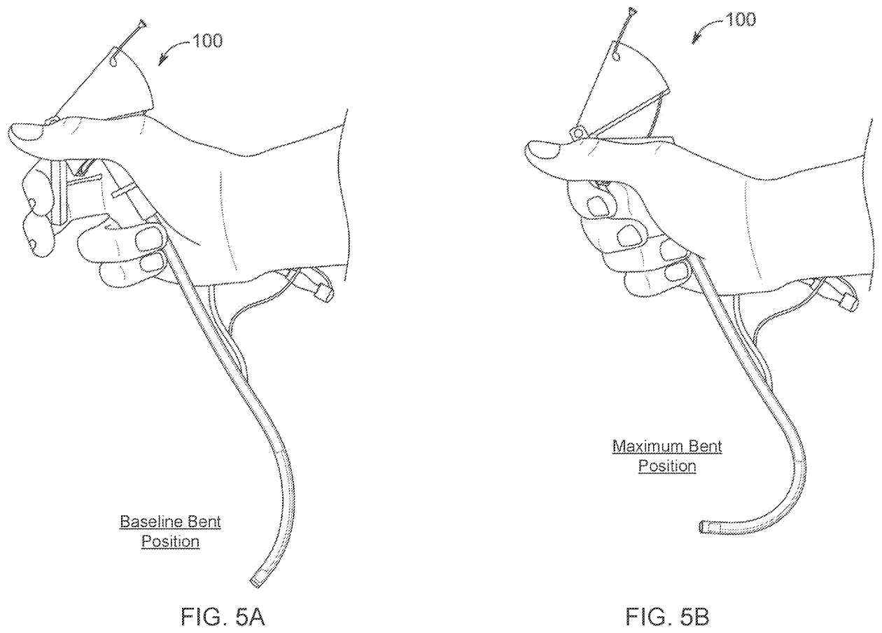

In some embodiments, as shown in FIG. 1, the articulation mechanism 150 includes a string 110 that is attached to the handle 120 at a first end of the string 110 and to the articulation mechanism 150 (e.g., elastically bendable element) at a second end of the string 110, such that, upon the movement of the handle 120, the string 110 causes a bending of the elastically bendable element along an exposed region of the articulation mechanism 150 (that is exposed outside of an opening (at the end point) of the shaft 140. In some embodiments, the string 110 comprises a durable and flexible polymer material, e.g., such as Kevlar or other polymer, fiber, or synthetic material to form a string or filament. In operations, the articulation mechanism 150 is actuated to bend in at least one plane by along a range of bending curves (also referred to as bent positions), i.e., a plurality of bent positions between a default bent position when a user has not activated (e.g., squeezed) the handle 120 and a maximum bent position when the user has fully activated the handle 120. FIGS. 5A and 5B (discussed later) show images depicting an example of the default (baseline) position and the maximum bent position, respectively.

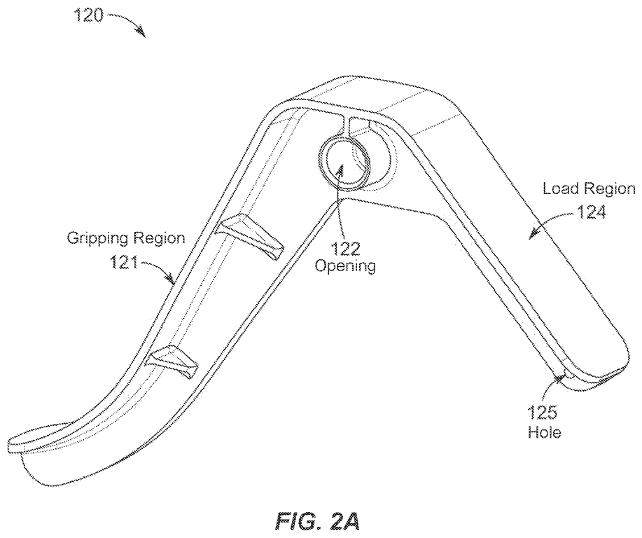

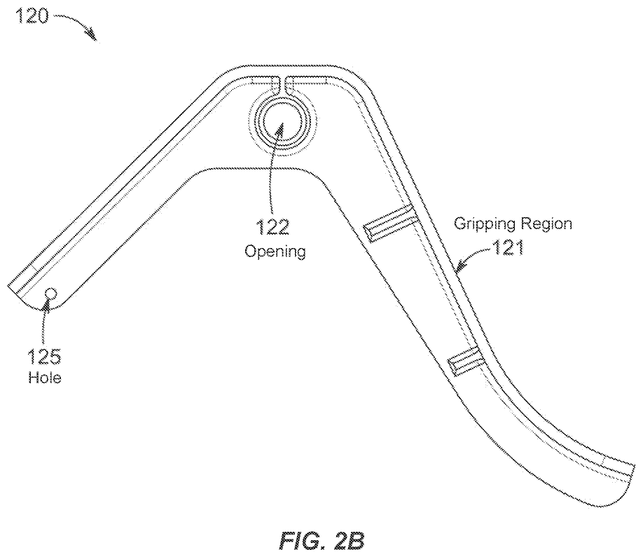

Referring to the example embodiment of the endotracheal tube stylet device 100 shown in FIG. 1, the housing 130 is structured to include an interior region within which a portion of the handle 120 resides. The handle 120 includes a handle connection point (hole 125, shown in FIGS. 2A and 2B) to which the string 110 is secured. In some embodiments, for example, the articulation mechanism 150 (e.g., elastically bendable element) includes an end point 160 that attaches the string 110 to the distal end of the articulation mechanism 150, such that the string 110 can be pulled via movement of the handle 120 to controllably move (e.g., bend) the articulation mechanism 150. Housing 130 is coupled to the shaft 140 by a removeable securement that can include, but is not limited to, one or more of a threaded (screw) interface, a snap-in interface, surface friction, pressure-fit, or other interface, or by a fixed securement that can include, but is not limited to, an adhesive or other. The shaft 140, also referred to herein as the “staple shaft,” can secure the proximal end (and/or region) of the elastically bendable element so that the region of the bendable element encased in the shaft 140 does not bend when the articulation mechanism 150 is actuated.

FIG. 2A shows a three-dimensional view of the handle 120, and FIG. 2B shows a two-dimensional frontal view of the handle 120. The handle 120 can be designed to be ergonomic for the user with respect to the stylet's use in a medical procedure. For example, the handle 120 (also referred to as “ergonomic handle”) can be configured to have a size and shape so that a user may easily grip the device 100 so that the user's fingers may surround the handle 120 and the user's palm may abut the opposite end of the housing 130, so that the user may hold the device 100 in the baseline position without moving the handle 120 and also apply simple squeezing motion on the handle 120 to cause the articulation mechanism 150 to bend in a manner directly proportional to the squeezing motion. For example, if a user squeezes the handle 120 to the maximum motion position of the handle, then the articulation mechanism 150 bends to its maximum bending position; and similarly, if the user squeezes the handle 120 to a half-way motion position of the handle, then the articulation mechanism 150 bends to substantially half-way bending position.

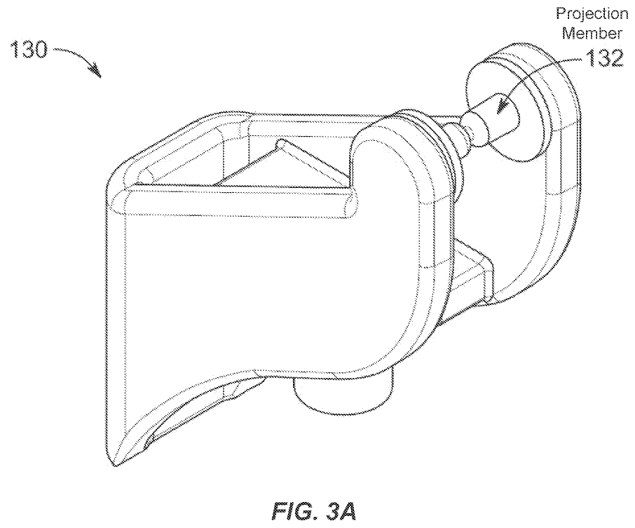

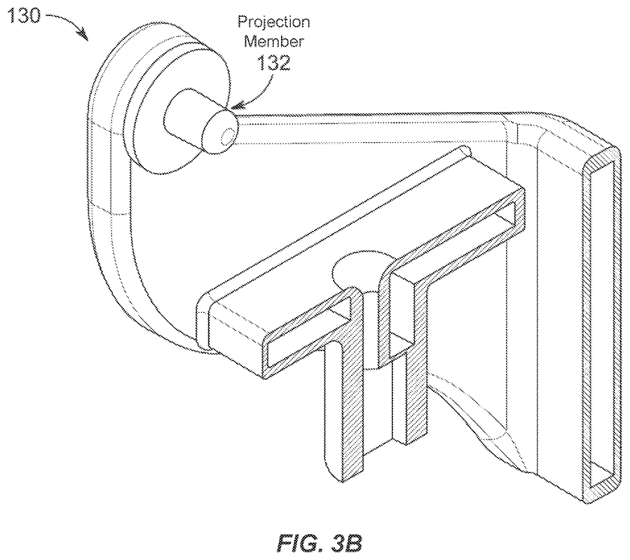

Ergonomic handle 120 has an opening 122 which can be used to couple with the housing 130 via the projection member 132 (shown in FIG. 3). The handle 120 can be configured as or to include a bent or curved lever arm, in which the opening 122 is positioned between two regions, i.e., a first region to allow a user to engage and a second region to actuate force onto the articulation mechanism 150, via the string. As illustrated in FIGS. 2A and 2B, the handle 120 has a gripping region 121 that a user can engage (e.g., grip) to cause a load region 124 of the handle 120 to rotate with respect to the opening 122, to generate mechanical force on the string that is connected at connection point 110 to the handle 120. In the example shown in FIGS. 2A and 2B, the string is connected to the handle 120 by securement to a hole 125. The generated mechanical force passes from the handle 120 situated in the housing 130 through the string to the articulation mechanism 150.

Referring back to FIG. 1, the shaft 140 has a first end that is coupled with the housing 130 and a second end that is coupled with the articulation mechanism 150. In the example of FIG. 1, the shaft 140 is a long tube that is made of a rigid material that restricts movement of the portion of the articulation mechanism 150 encased within the shaft 140; whereas in some embodiments, the shaft 140 can be made of a semi-flexible material to allow some bending of the shaft (if desired for certain applications). The shaft 140 is made of material that can restrict (or at least partially restrict) movement of the proximal regions of the articulation mechanism 150 proximate to the housing 130, while allowing the articulation mechanism 150 bend to a controllable degree at the distal regions of the articulation mechanism 150 that have exited the shaft 140. For various embodiments, the shaft 140 can be designed in various lengths, which affects the maximum bending degree of the articulation mechanism 150 bends and the radius of curvature. For example, the shorter the length of the shaft 140 for a given-length elastically bendable element (e.g., bendable metal spring), the greater the maximum bending degree of articulation mechanism 150 and the larger the radius of curvature; the longer the length of a shaft for the given-length bendable element, the lesser the maximum bending degree of the articulation mechanism 150 and the shorter the radius of curvature. In some embodiments, the length of the rigid shaft 140 can be adjustable by the user, e.g., such that the aperture from which the articulation mechanism 150 is exposed is brought further or closer to the interface where the shaft 140 couples with the housing 130.

In some example embodiments, the articulation mechanism 150, like that shown in the example of FIG. 1, has a baseline position, which can have a certain bending degree to one side. In some embodiments of the device 100, for example, the device 100 is configured to allow the user to adjust this baseline position based on need. In some embodiments, for example, the articulation mechanism 150 can include a guide to maintain the string 110 to run alongside the elastically bendable element (e.g., bendable metal spring) during actuation of the device 110 (e.g., during movement of the handle 120 to pull the string 110). For example, the guide may include an endotracheal tube that the endotracheal tube stylet device 100 will be inserted within prior to a medical procedure. Yet, in some embodiments, for example, the guide is configured as a coating that encompasses the elastically bendable element and string 110 (e.g., at least the exposed portion outside of the shaft). For example, the guide can include a silicone or plastic coating. In any embodiments of the guide, the string 110 can be pulled by actuation of the device 100 to cause the bending of the articulation mechanism 150.

FIGS. 3A and 3B show schematics depicting an example embodiment of the housing 130 for example endotracheal tube style device 100. FIG. 3A shows a perspective view of the housing 130, and FIG. 3B shows a schematic illustrating a cross-sectional view of the housing 130. In some embodiments of the endotracheal tube style device 100, the housing 130 includes two projection members 132 projecting inwardly from the interior sides of the housing 130 (shown in FIG. 3), in which the projection members 132 are operable to couple with the handle 120 via an opening 122 of the handle 120 (shown in FIG. 2A and 2B). In such configurations of the housing 130, a first arm of the handle 120 that connects the string 110 (e.g., at hole 125) is disposed at least partially within the housing 130, and a second arm of the handle 120 that provides the gripping region 121 is disposed outside of the housing 130, where the handle 120 can pivotably move about the projection members 132 based on user control of the handle 120.

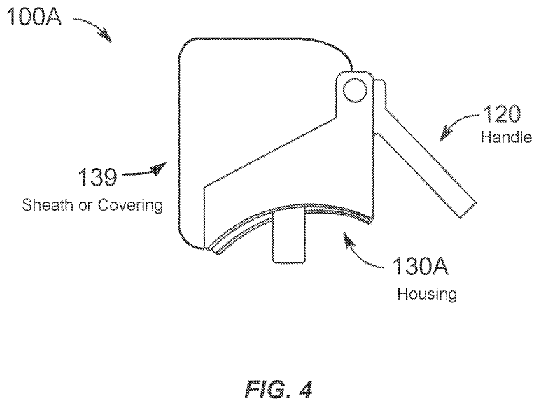

FIG. 4 shows a diagram of an example embodiment of the endotracheal tube stylet device 100 shown in FIG. 1, labeled device 100A, featuring another example embodiment of the housing 130, labeled housing 130A. The housing 130A includes a sheath or covering 139 to cover the interior of the housing 130A where the handle 120 is operable to move within. For example, the sheath or covering 139 can be removable from the housing 130A to allow access to view and/or manipulate the interface or connection of the handle 120 with the string 110. Also, for example, the sheath or covering 139 can provide additional or augmented surface area to facilitate contact with the hand of the user, e.g., such as the palm region of the hand. In various embodiments, the sheath or covering 139 can be configured of various sizes to accommodate different sized hands and/or be configured of various materials to provide surfaces with different friction properties. For example, the additional or augmented surface area includes an underlying cushion, perforations, and/or ridges to increase comfort and/or friction of the device 100A.

FIGS. 5A and 5B show images of an example endotracheal tube stylet where a user adjusts the tip of the endotracheal tube through controlled bending of the articulation mechanism 150. In the example, a user first inserts the endotracheal tube stylet device into a standard endotracheal tube (not shown). As shown in FIG. 5A, the endotracheal tube stylet is in its default or baseline (or default) position before the user actuates bending of the stylet. From this baseline position, the articulation mechanism 150 is actuated to bend with a certain degree. FIG. 5B shows an adjusted maximum bent position after the user squeezes the handle 120 of the stylet device 100 to the maximum motion position of the handle 120. In the example shown in FIG. 5B, the maximum bent position has a higher bend degree than in the baseline position. In implementations, for example, if a user squeezes the handle 120 to a motion position less than the maximum bending degree of the handle, then the articulation mechanism 150 bends to a position between the baseline position and the maximum bent position. In some instances, for example, a user may overbend the articulation mechanism 150 during a medical procedure and subsequently realize he/she wants to lessen the bend degree; and thus, in such instances, the user can partially release the squeeze of the handle 120 which will cause the articulation mechanism 150 curvature to reduce back toward the baseline position.

In some implementations, for example, a user may maintain the current bent position of the articulation mechanism 150 by manually holding the handle 120 in the certain position corresponding to the current bent position of the articulation mechanism 150. Whereas, in some example embodiments, the endotracheal tube stylet device 100 can include a lock mechanism that controllably fixes the articulation mechanism 150 to stay in the present position when the lock mechanism is engaged, and allows the user to unlock the position to allow for the articulation mechanism 150 to bend in any of the bending positions between the baseline position and the maximum bent position.

As discussed above, the endotracheal tube stylet device 100 can be used in medical procedures involving intubation. In an example medical procedure, after insertion of the stylet device in the endotracheal tube, the user can select a laryngoscope of choice (for visualization of the airway during an intubation process) and insert the endotracheal stylet/tube into the patient's mouth under direct visualization. For adjusting the bending of the tube, the user can pull the ergonomic handle (e.g., trigger of the stylet) with one or more of his/her fingers, which causes the tube to curve anteriorly to reach the airway. After the endotracheal tube is inserted into the airway as guided by the endotracheal tube stylet device, the stylet can be safely removed.

For example, in some embodiments in accordance with the present technology, a method for endotracheal intubation includes providing an endotracheal tube stylet (e.g., which can comprise a housing, a handle that is operably coupled to the housing, a shaft that is coupled to the housing, and an articulation mechanism that is operably coupled to the handle and able to bend in a plurality of bending positions upon movement of the handle; inserting the endotracheal tube stylet into a endotracheal tube; inserting the endotracheal tube-surrounded endotracheal tube stylet into an airway of a subject; moving the handle of the endotracheal tube stylet to adjust the articulation mechanism to be in a bent position among the plurality of bending positions; and removing the endotracheal tube stylet from the endotracheal tube after the endotracheal tube is inserted in the airway as guided by the endotracheal tube stylet. In some implementations of the method, for example, the subject includes a non-living model of an animal or a living animal. In some implementations of the method, for example, the bent position of the articulation mechanism is between a baseline bent position and a maximum bent position. In some implementations of the method, for example, the articulation mechanism is configured to move towards the maximum bent position through squeezing the handle. In some implementations of the method, for example, the articulation mechanism is configured to move back towards the baseline bent position through releasing the handle.

In these or other implementations, the bending of the articulation mechanism 150 enables the endotracheal tube to bend in the same degree with the endotracheal tube stylet device 100. Various embodiments of the endotracheal tube stylet 100 can be configured to bend to a desired degree. The bending of the endotracheal tube prevents pressure from being placed on the posterior aspect of the larynx, thereby preventing injury to the patient.

### EXAMPLES

In some example embodiments (example 1) in accordance with the present technology, an endotracheal tube stylet apparatus includes a housing; a handle that is coupled to the housing; a shaft that is coupled to the housing; and an articulation mechanism comprising a bendable element and a string, the articulation mechanism extending from the housing through and out of an aperture of the shaft and operable to bend upon control by movement of the handle, wherein the string is coupled to the handle and to a distal end of the bendable element, such that, upon the movement of the handle, the string is pulled to cause a bending of the bendable element along an exposed region of the articulation mechanism outside of the aperture of the shaft.

Example 2 includes the endotracheal tube stylet apparatus of any of examples 1-15, wherein the handle comprises a segment having a first region that is at least partially encased within the housing and a second region that is disposed outside the housing to allow a user to grip the handle, the segment having a hole at a portion encased within the housing, and wherein the housing comprises a projection member structured to project inwardly from an interior side of the housing and pass through the hole of the segment to allow the handle to rotate with respect to the projection member.

Example 3 includes the endotracheal tube stylet apparatus of any of examples 1-15, wherein the handle comprises a handle connection point structured to secure the string.

Example 4 includes the endotracheal tube stylet apparatus of any of examples 1-15, wherein the articulation member further comprises a guide to maintain the string to follow a trajectory of the bending of the bendable element (e.g., bendable metal spring and/or bendable polymer) when the articulation mechanism is controlled to move.

Example 5 includes the endotracheal tube stylet apparatus of example 4 or any of examples 1-15, wherein the guide includes an outer coating that surrounds the string and the bendable element at least along a portion of the exposed region of the articulation mechanism.

Example 6 includes the endotracheal tube stylet apparatus of example 4 or any of examples 1-15, wherein the guide includes an endotracheal tube.

Example 7 includes the endotracheal tube stylet apparatus of any of examples 1-15, wherein the bendable element (e.g., bendable metal spring and/or bendable polymer) of the articulation mechanism further comprising a connection point to secure the string.

Example 8 includes the endotracheal tube stylet apparatus of any of examples 1-15, wherein the string comprises Kevlar or other polymer, fiber, or synthetic material.

Example 9 includes the endotracheal tube stylet apparatus of any of examples 1-15, wherein the shaft is made of material that can restrict movement of the articulation mechanism. Example 10 includes the endotracheal tube stylet apparatus of any of examples 1-15,

wherein the articulation mechanism is actuatable to bend in at least one plane along a range of bending positions.

Example 11 includes the endotracheal tube stylet apparatus of any of examples 1-15, wherein the articulation mechanism is configured in a default bent position when the handle is in a default position with respect to the housing, and wherein the articulation mechanism is actuatable to bend in a maximum bent position when the handle is moved to a maximum position with respect to the housing.

Example 12 includes the endotracheal tube stylet apparatus of any of examples 1-15, wherein the bendable element comprises a bendable metal spring or bendable polymer; and/or where the bendable element is configured as a rod, tube, or other elongated component.

Example 13 includes the endotracheal tube stylet apparatus of any of examples 1-15, further comprising a sheath or cover to cover at least a portion of the housing.

Example 14 includes the endotracheal tube stylet apparatus of example 13 or any of examples 1-15, wherein the sheath or cover is removable from the housing to allow access to an interface of the handle with the string.

Example 15 includes the endotracheal tube stylet apparatus of example 13 or any of examples 1-14, wherein the sheath or covering includes an outer surface area to facilitate contact with the hand of the user, wherein the outer surface area includes one or more of an underlying cushion, perforations, or ridges.

In some example embodiments (example 16) in accordance with the present technology, a method for endotracheal intubation includes providing an endotracheal tube stylet that comprises a housing, a handle that is operably coupled to the housing, a shaft that is coupled to the housing, and an articulation mechanism that is operably coupled to the handle and able to bend in a plurality of bending positions upon movement of the handle; inserting the endotracheal tube stylet into a endotracheal tube; inserting the endotracheal tube that surrounds the endotracheal tube stylet into an airway of a subject; moving the handle of the endotracheal tube stylet to adjust the articulation mechanism to be in a bent position among the plurality of bending positions; and removing the endotracheal tube stylet from the endotracheal tube after the endotracheal tube is inserted in the airway as guided by the endotracheal tube stylet.

Example 17 includes the method of any of examples 16-20, wherein the subject includes a non-living model of an animal or a living animal.

Example 18 includes the method of any of examples 16-20, wherein the bent position of the articulation mechanism is between a baseline bent position and a maximum bent position.

Example 19 includes the method of example 18 or any of examples 16-20, wherein the articulation mechanism is configured to move towards the maximum bent position through squeezing the handle.

Example 20 includes the method of example 18 or any of examples 16-19, wherein the articulation mechanism is configured to move back towards the baseline bent position through releasing the handle.

In some example embodiments (example 21) in accordance with the present technology, an endotracheal tube stylet apparatus includes a frame; a handle that is moveably coupled to the frame; and an articulation mechanism that is at least partially disposed outside of the frame and operable to bend upon control by movement of the handle.

Example 22 includes the endotracheal tube stylet apparatus of any of examples 21-25, wherein the articulation mechanism comprises a bendable elongated element that is operable to bend along a bending region of the bendable elongated element outside of the frame upon the control by movement of the handle.

Example 23 includes the endotracheal tube stylet apparatus of example 22 or any of examples 21-25, wherein the bendable elongated element includes a bendable metal spring and/or a bendable polymer.

Example 24 includes the endotracheal tube stylet apparatus of example 22 or any of examples 21-25, wherein the articulation mechanism comprises a string, wherein the string is coupled to the handle and to a distal end of the bendable elongated element, such that, upon the movement of the handle, the string is pulled to cause a bending of the bendable elongated element along the bending region of the articulation mechanism outside of the frame.

Example 25 includes the endotracheal tube stylet apparatus of any of examples 21-24, wherein the articulation member comprises a guide to cover and/or maintain components of the articulation member to bend along a bending trajectory of the articulation mechanism.

In some example embodiments (example 26) in accordance with the present technology, an endotracheal tube stylet apparatus includes a housing; a handle that is coupled to the housing; a shaft that is coupled to the housing; and an articulation mechanism comprising a bendable element and a string that is coupled to the handle and to a distal end of the bendable element, the articulation mechanism extending from the housing through and out of an aperture of the shaft and operable to bend along a bending region of the bendable element outside of the shaft upon control by movement of the handle.

Example 27 includes the endotracheal tube stylet apparatus of example 26, wherein the endotracheal tube stylet apparatus includes one or more features from any of examples 1-15.

In some example embodiments (example 28) in accordance with the present technology, a system for endotracheal intubation and interrogation includes an endotracheal tube stylet apparatus as in any of examples 1-15, 21-25, and/or 26-27; and a standard laryngoscope and/or a video laryngoscope.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described, and other implementations, enhancements and variations can be made based on what is described and illustrated in this patent document.

1. An endotracheal tube stylet apparatus, comprising:
a housing;
a handle that is coupled to the housing;
a shaft that is coupled to the housing; and
an articulation mechanism comprising a bendable element and a string, the articulation mechanism extending from the housing through and out of an aperture of the shaft and operable to bend upon control by movement of the handle,
wherein the string is coupled to the handle and to a distal end of the bendable element, such that, upon the movement of the handle, the string is pulled to cause a bending of the bendable element along an exposed region of the articulation mechanism outside of the aperture of the shaft.

2. The endotracheal tube stylet apparatus of claim 1, wherein the handle comprises a segment having a first region that is at least partially encased within the housing and a second region that is disposed outside the housing to allow a user to grip the handle, the segment having a hole at a portion encased within the housing, and wherein the housing comprises a projection member structured to project inwardly from an interior side of the housing and pass through the hole of the segment to allow the handle to rotate with respect to the projection member.

3. The endotracheal tube stylet apparatus of claim 1, wherein the handle comprises a handle connection point structured to secure the string.

4. The endotracheal tube stylet apparatus of claim 1, wherein the articulation member further comprises a guide to maintain the string to follow a trajectory of the bending of the bendable element when the articulation mechanism is controlled to move.

5. The endotracheal tube stylet apparatus of claim 4, wherein the guide includes an outer coating that surrounds the string and the bendable element at least along a portion of the exposed region of the articulation mechanism.

6. The endotracheal tube stylet apparatus of claim 4, wherein the guide includes an endotracheal tube.

7. The endotracheal tube stylet apparatus of claim 1, wherein the bendable element of the articulation mechanism further comprising a connection point to secure the string.

8. The endotracheal tube stylet apparatus of claim 1, wherein the string comprises Kevlar or other polymer, fiber, or synthetic material.

9. The endotracheal tube stylet apparatus of claim 1, wherein the shaft is made of material that can restrict movement of the articulation mechanism.

10. The endotracheal tube stylet apparatus of claim 1, wherein the articulation mechanism is actuatable to bend in at least one plane along a range of bending positions.

11. The endotracheal tube stylet apparatus of claim 1, wherein the articulation mechanism is configured in a default bent position when the handle is in a default position with respect to the housing, and wherein the articulation mechanism is actuatable to bend in a maximum bent position when the handle is moved to a maximum position with respect to the housing.

12. The endotracheal tube stylet apparatus of claim 1, wherein the bendable element comprises a bendable metal spring or bendable polymer.

13. The endotracheal tube stylet apparatus of claim 1, further comprising a sheath or cover to cover at least a portion of the housing.

14. The endotracheal tube stylet apparatus of claim 13, wherein the sheath or cover is removable from the housing to allow access to an interface of the handle with the string.

15. The endotracheal tube stylet apparatus of claim 13, wherein the sheath or covering includes an outer surface area to facilitate contact with the hand of the user, wherein the outer surface area includes one or more of an underlying cushion, perforations, or ridges.

16. A method for endotracheal intubation, comprising:
providing an endotracheal tube stylet that comprises a housing, a handle that is operably coupled to the housing, a shaft that is coupled to the housing, and an articulation mechanism that is operably coupled to the handle and able to bend in a plurality of bending positions upon movement of the handle;
inserting the endotracheal tube stylet into a endotracheal tube;
inserting the endotracheal tube that surrounds the endotracheal tube stylet into an airway of a subject;
moving the handle of the endotracheal tube stylet to adjust the articulation mechanism to be in a bent position among the plurality of bending positions; and
removing the endotracheal tube stylet from the endotracheal tube after the endotracheal tube is inserted in the airway as guided by the endotracheal tube stylet.

17-20. (canceled)

21. An endotracheal tube stylet apparatus, comprising:
a frame;
a handle that is moveably coupled to the frame; and
an articulation mechanism that is at least partially disposed outside of the frame and operable to bend upon control by movement of the handle.

22. The endotracheal tube stylet apparatus of claim 21, wherein the articulation mechanism comprises a bendable elongated element that is operable to bend along a bending region of the bendable elongated element outside of the frame upon the control by movement of the handle.

23. (canceled)

24. The endotracheal tube stylet apparatus of claim 22, wherein the articulation mechanism comprises a string, wherein the string is coupled to the handle and to a distal end of the bendable elongated element, such that, upon the movement of the handle, the string is pulled to cause a bending of the bendable elongated element along the bending region of the articulation mechanism outside of the frame, and/or wherein the bendable elongated element includes a bendable metal spring and/or a bendable polymer.

25. The endotracheal tube stylet apparatus of claim 21, wherein the articulation member comprises a guide to cover and/or maintain components of the articulation member to bend along a bending trajectory of the articulation mechanism.
